(19) United States
(12) Patent Application Publication
(10) Pub. No.: US 2010/0151451 A1
(43) Pub. Date: Jun. 17, 2010

(54) Method for Identifying the Genotype in Position 171 of the Ovine Prion Protein as well as Kits for Implementing said Method

(75) Inventors: Jacques Grassi, Bures sur Yvette (FR); Nathalie Morel, Gentilly (FR); Jean-Marc Gilles Bilheude, Hardricourt (FR); Juan-Maria Torres Trillo, Valdetorres de Jarama (ES); Alejandro Brun Torres, Madrid (ES)

Correspondence Address: MCDONNELL BOEHNEN HULBERT & BERGHOFF LLP, 300 S. WACKER DRIVE, 32ND FLOOR, CHICAGO, IL, 60606, US

(73) Assignees: BIO-RAD PASTEUR, Marnes La Coquette (FR); COMMISSARIAT a l'ENERGIE ATOMIQUE, Paris (FR)

(21) Appl. No.: 12/095,058

(22) PCT Filed: Nov. 24, 2006
(86) PCT No.: PCT/FR03/51230

§ 371 Date: Aug. 15, 2008

(30) Foreign Application Priority Data
Nov. 25, 2005 (FR) 0511937

(51) Int. Cl. C12Q 1/68

(52) U.S. Cl. 435/6

(57) ABSTRACT

The invention relates a method to identify genotype at position 171 of the ovine PrP, and a method to select an ovine for reproduction.

It also relates kits to implement these methods.

The identification method of the invention comprises the steps of treating a sample of ovine biological fluid to be tested containing the PrP with a denaturing and reducing solution, immobilizing the denatured and reduced PrP, optionally via a ligand, on a solid phase, contacting the denatured, reduced and immobilized PrP with at least one detection antibody, and of detecting the possible presence of said at least one detection body, one of the ligand or of said at least one detection antibody specifically binding with the PrP having a particular allelic form at position 171.

The invention finds particular application in the medical field, more particularly veterinary and sanitary fields.

The invention relates a method to identify or analyse the genotype at position 171 of the ovine prion protein, called PrP, and kits to implement this method.

It also relates a method allowing the screening of a population of ovines in relation to their resistance to transmissible subacute spongiform encephalopathies (TSSE).

It also relates a method allowing the screening of a population of ovines on the basis of its sensitivity to TSSE.

### PRIOR ART

Scrapie is a disease affecting ovines which has been known in Europe for over two centuries. Its particular signs are social behavioural disorders of the animal, which has a tendency to remain alone, eating disorders and locomotive disorders such as the onset of trembling and stiffness of its hind parts. Other signs generally include pruritus and loss of wool.

Work focusing on the experimental transmission of sheep scrapie started in 1938 on British sheep, and greatly contributed towards showing the involvement of genetic factors in the transmission of the disease.

The exact nature of the agent involved in scrapie as in other transmissible subacute spongiform encephalopathies (TSSEs) has not yet been definitely established, but today is given near-overall scientific consensus based on extensive experimental evidence. The transmission agent responsible for these diseases appears to be a protein of the host, PrP (for prion protein) in an abnormal form that is partly resistant to proteolytic digestion, commonly called PrPres for “prion-protein resistant”. The infectious property associated with these abnormal prion proteins has proved to be extremely resistant to known inactivation methods used to inactivate <<conventional>> pathogenic microorganisms (bacteria, viruses, yeasts).

The gene of the ovine PrP is carried by chromosome 13 and encodes a polypeptide of 256 amino acids.

It has long been known that the natural occurrence of scrapie in ovines is related to the polymorphism of the PrP gene, and in particular to the polymorphisms of codons 136, 154 and 171 of this protein.

Several alleles (or allotypes) of PrP have been described, the chief ones being the alleles ARQ, VRQ, ARR, AHQ and ARH. The combination of two of these alleles can produce a large number of different genotypes. According to the British National Scrapie Plan (NSP) the majority of the sheep population carries one of fifteen genotypes, listed below, on codons 136, 154 and 171:

A₁₃₆R₁₅₄R₁₇₁/A₁₃₆R₁₅₄R₁₇₁, denoted ARR/ARR

A₁₃₆R₁₅₄R₁₇₁/A₁₃₆H₁₅₄Q₁₇₁, denoted ARR/AHQ

A₁₃₆R₁₅₄R₁₇₁/A₁₃₆R₁₅₄H₁₇₁, denoted ARR/ARH

A₁₃₆R₁₅₄R₁₇₁/A₁₃₆R₁₅₄Q₁₇₁, denoted ARR/ARQ

A₁₃₆H₁₅₄Q₁₇₁/A₁₃₆H₁₅₄Q₁₇₁, denoted AHQ/AHQ

A₁₃₆H₁₅₄Q₁₇₁/A₁₃₆R₁₅₄H₁₇₁, denoted AHQ/ARH

A₁₃₆H₁₅₄Q₁₇₁/A₁₃₆R₁₅₄Q₁₇₁, denoted AHQ/ARQ

A₁₃₆R₁₅₄H₁₇₁/A₁₃₆R₁₅₄H₁₇₁, denoted ARH/ARH

A₁₃₆R₁₅₄H₁₇₁/A₁₃₆R₁₅₄Q₁₇₁, denoted ARH/ARQ

A₁₃₆R₁₅₄Q₁₇₁/A₁₃₆R₁₅₄Q₁₇₁, denoted ARQ/ARQ

A₁₃₆R₁₅₄R₁₇₁/V₁₃₆R₁₅₄Q₁₇₁, denoted ARR/VRQ

A₁₃₆H₁₅₄Q₁₇₁/V₁₃₆R₁₅₄Q₁₇₁, denoted AHQ/VRQ

A₁₃₆R₁₅₄H₁₇₁/V₁₃₆R₁₅₄Q₁₇₁, denoted ARH/VRQ

A₁₃₆R₁₅₄Q₁₇₁/V₁₃₆R₁₅₄Q₁₇₁, denoted ARQ/VRQ

V₁₃₆R₁₅₄Q₁₇₁/V₁₃₆R₁₅₄Q₁₇₁, denoted VRQ/VRQ.

More simply, sheep can be classified into 3 groups in relation to their genotype and their resistance to TSSEs:
resistant animals which are not (or only extremely rarely) infected, even when they are placed in an environment having a high occurrence of the disease, or which show very high resistance under experimental infections. These are animals having genotypes ARR/ARR. It is to be noted however that the recent discovery of atypical cases of scrapie also affecting the ARR/ARR genotype has partly called into question the near-absolute resistance that is routinely associated with this genotype.
highly sensitive animals showing a high incidence of the disease with short incubation times. These are animals having genotypes VRQ/VRQ (the genotype deemed to be the most sensitive), ARQ/VRQ and ARQ/ARQ, and
animals which can show variable incidence of the disease and after a long period of incubation. These are animals having the other genotypes.

For a long time, plans to combat scrapie have been based on total slaughter of all infected flocks. It has been ascertained however that those animals re-introduced afterwards generally become infected. This situation is probably connected with the strong persistence of the contagious agent in the environment of bred animals, and its resistance to decontamination techniques. In the USA, eradication campaigns have been carried out since 1952, and from a global viewpoint have not been crowned with success. Solely the policy led by Iceland appears to produce good results in this respect. It is a highly costly and constraining policy however. It is particularly based on dividing the territory into affected areas and healthy areas, total slaughter of infected flocks with destruction of all breeding equipment, removing soil around the breeding area and forbidding re-introduction of animals into the said area for five years. Evidently, the animals used to repopulate “cleansed” areas are taken from non-affected areas.

The selection of homozygous animals carrying the A₁₃₆R₁₅₄R₁₇₁ allele, even if it cannot be considered as the absolute solution to eradicate transmissible spongiform encephalopathies (TSEs) in ovine flocks, remains a most appropriate strategy however for controlling propagation of most TSE strains including the agent of bovine spongiform encephalopathy (BSE) and is of obvious economic interest.

Great Britain, France and the Netherlands for example have already started implementing plans to enrich sheep populations with resistant animals for the purpose of combating scrapie.

The French plan particularly comprises the elimination of animals carrying the V₁₃₆R₁₅₄Q₁₇₁ allele associated with maximum sensitivity. Its high frequency among the population is a major risk factor for propagation of the disease. Therefore a sheep carrying this allele is considered to be a “non-authorized” animal. As such, it cannot be sold for reproduction purposes. The indication <<scrapie-sensitive genotype>> is given on the certificated issued by the Union for promoting animal races (Union Pour la Promotion des Races Animales—UPRA) or by any other similar body.

Said objective requires identification of the PrP genotype of sheep. A certain number of methods based on molecular biology have already been put forward for this purpose. Several methods using PCR can be cited, a so-called pyrosequencing method and a method allowing simple colorimetric determination after single amplification of the PrP gene by PCR.

However, these methods which all involve PCR amplification have to be carried out by specialized laboratories and are very costly, which represents a true obstacle for their large-scale use.

It has been seen that the A₁₃₆R₁₅₄R₁₇₁/A₁₃₆R₁₅₄R₁₇₁ genotype is the genotype it is desired to select for an animal intended to be used for reproduction.

It is the only genotype, from among the fifteen known genotypes, which has two R amino acids at positions 171 of the PrP. This genotype is denoted R/R.

As we have already seen it is the V₁₃₆R₁₅₄Q₁₇₁/V₁₃₆R₁₅₄Q₁₇₁ genotype which it is sought to avoid in reproduction animals.

### OBJECTS OF THE INVENTION

The object of the present invention is to solve the new technical problem which is the provision of a method to identify the genotype at position 171 of the ovine prion protein (PrP).

It is also the object of the invention to solve the new technical problem which is to provide a kit to identify the genotype at position 171 of the ovine PrP.

One particular object of the invention is to solve the new technical problem consisting of providing a method to identify the genotype at position 171 and/or an identification kit for this genotype to allow sheep breeders to select, choose, or identify resistant or sensitive animals, and in particular to discriminate resistant animals from sensitive animals or conversely.

One particular object of the invention is to solve these technical problems under a reproduction plan to eradicate or at least reduce the presence of the prion in ovines.

One particular object of the invention is to solve this technical problem in order to combat sheep crapie.

One particular object of the invention is to solve these technical problems using a method other than a method of PCR or Western Blot type, these latter methods being costly and requiring specialized laboratory equipment.

Therefore, a further object of the invention is to solve these technical problems using a rapid test which notably does not require any specialized equipment other than that used by a conventional analysis laboratory.

A particular object of the invention is to solve these technical problems in reproducible, industrial, reliable, swift manner and at lowest cost.

### DESCRIPTION OF THE INVENTION

It has now been discovered that it is possible to identify the amino acid present at position 171 of the ovine PrP through the use of at least one antibody which specifically recognizes certain allelic forms of the PrP.

This method can be applied to a biological fluid of sheep, such as the plasma, blood, serum, milk, saliva and urine.

Also, this method is easy to carry out and is quick and economic.

For this purpose, the invention proposes a method to identify or analyze the genotype at position 171 of the ovine PrP comprising the steps of:

a) treating the ovine biological fluid to be tested containing the PrP with a denaturing and reducing solution,

b) immobilizing, optionally via a ligand, the denatured, reduced PrP on a solid phase,

c) contacting the denatured, reduced, immobilized PrP with at least one detection antibody, and

d) detecting the possible presence of said at least one detection antibody,

and

in which one of the ligand or of said at least one detection antibody specifically binds to the PrP having a particular allelic form at position 171.

Preferably the denaturing and reducing solution comprises:

a) at least one denaturing agent chosen from among:
a surfactant chosen from the group consisting of:
anionic surfactants, such as SDS (sodium dodecylsulfate), sarcosyl (lauroyl sarcosine), sodium cholate, sodium glycocholate, sodium deoxycholate, sodium taurocholate, sodium caprylate, sodium 1-decanesulfonate, sodium laurylsulfate and lithium laurylsulfate;
zwitterionic surfactants, such as SB 3-10 (decyl-sulfobetaine), SB 3-12 (dodecyl-sulfobetaine), SB 3-14 (tetradecyl-sulfobetaine), SB 3-16 (hexadecyl-sulfobetane), SB 3-18 (octadecyl-sulfobetaine), CHAPS and CHAPSO et deoxy CHAPS;
non-ionic surfactants, such as Triton X-100, Triton X-114, Tween 20, Tween 80, Brij 35 (polyoxyethylene 23 laurylether), nonidet P-40, n-decyl-beta-D-glucopyranoside, n-dodecyl-beta-D-glucopyranoside, n-octyl-beta-D-glucopyranoside, n-octyl-alpha-D-glucopyranoside;
their mixtures, and/or
   a chaotropic agent, and

b) at least one reducing agent.

The chaotropic agent is chosen from among urea, guanidine, guanidine hydrochloride, guanidine thiocyanate or one of their mixtures.

More preferably, the denaturing and reducing solution comprises a mixture of ionic surfactants, in particular anionic surfactants, and more particularly of SDS and sarcosyl.

More precisely, the denaturing and reducing solution contains at least 0.5 wt. % of surfactant relative to the total volume of the mixture consisting of the sample to be treated and the denaturing and reducing solution, and in particular is equal to or more than 2 wt. % relative to the total volume of the mixture.

Preferably, the at least one reducing agent is chosen from the group consisting of DTT (dithiothreitol), TCEP (Tris(2-carboxyethyl)phosphine hydrochloride), DTE (dithio erythritol), Beta mercaptoethanol, 2-mercaptoethylamine or one of their mixtures.

In this case, preferably the concentration of reducing agent comprises between 2.5 mM and 100 mM, in particular between 5 mM and 50 mM, and more particularly between 5 mM and 30 mM in the mixture consisting of the sample to be treated and the denaturing and reducing solution.

Most preferably, the denaturing and reducing solution comprises a mixture of sarcosyl, SDS and DTT.

If the denaturing and reducing solution comprises a chaotropic agent, the concentration of this chaotropic agent is preferably equal to or more than 1 M, and more particularly equal to or more than 3 M. If the chaotropic agent is urea, the concentration is preferably 8 M.

In a first embodiment of the method of the invention to identify the genotype at position 171 of the ovine PrP, the denatured and reduced PrP is immobilized via a ligand which is a capture antibody capable of retaining the PrP by affinity binding, and the detection antibody is an antibody specifically binding to the PrP having a particular allelic form at position 171, this position being different from the epitopic site recognized by the capture antibody.

In a second embodiment of the method of the invention, the denatured and reduced PrP is immobilized via a ligand which is a capture antibody specifically binding to the PrP having a particular allelic form at position 171 and the detection antibody is an antibody capable of binding to the PrP by affinity binding at an epitopic site different to the one recognized by the capture antibody.

In a third embodiment of the method of the invention, the denatured and reduced PrP is immobilized via a ligand chosen from among the following molecules: plasminogen, avidine, streptavidine, glycose aminoglycans, hesperidine, porphyrins, streptamycine and tetracycline, and the detection antibody is an antibody specifically binding to the PrP having a particular allelic form at position 171.

In a fourth embodiment of the method of the invention, the denatured and reduced PrP is directly immobilized on the solid phase, and the detection antibody is an antibody specifically binding to the PrP having a particular allelic form at position 171.

In all the embodiments of the method of the invention, preferably the solid phase is chosen from among microtiter plates, beads, tubes, in polymer notably in polystyrene, polyethylene or latex. Preferably, the solid phase is a microtiter plate in polystyrene.

Also, in all the embodiments of the method of the invention, preferably the sample of biological fluid is chosen from among blood, plasma, serum or milk.

In the first embodiment of the method of the invention, the capture antibody is an antibody which binds with the PrP without any competition from the binding of the detection antibody with the PrP which is specific to allelic form at position 171. In particular this antibody may be chosen from among the antibodies SAF-15, SAF-31, SAF-32, SAF-33, SAF-34, SAF-35, SAF-37, 3B5, SAF-84, SHA-31, BAR-222, BAR-224, BAR-233, and 8G8.

As capture antibody preference is given to an antibody targeting the octa repeat region of the Prp, namely: SAF-15, SAF-31, SAF-32, SAF-33, SAF-34, SAF-35, SAF-37, and 3B5.

In this case, the detection antibody is preferably chosen from among the labeled 2A11 antibody, the labeled 11C6 antibody, the labeled BAR226 antibody and the labeled 12F10 antibody.

However, preferably in the first embodiment of the method of the invention, the capture antibody is the SAF-34 antibody or the 3B5 antibody, and the detection antibody is the labeled 2A11 antibody.

Advantageously, in the second embodiment, the ligand is a capture antibody chosen from among the antibodies 2A11, 11C6, BAR-226, and 12F10.

Advantageously, the detection antibody is chosen from among SAF-15, SAF-31, SAF-32, SAF-33, SAF-34, SAF-35, SAF-37, 3B5, SAF-84, SHA-31, BAR-222, BAR-224, BAR-233, and 8G8.

Preferably, in the second embodiment of the method of the invention, the capture antibody can be chosen from among the antibodies 2A11, 12F10, BAR226, 11C6, and the detection antibody is chosen from among the labeled antibodies SAF-15, SAF-31, SAF-32, SAF-33, SAF-34, SAF-35, SAF-37, 3B5, BAR-224, SHA-31, and 8G8.

The invention also proposes a method to screen an ovine population in relation to its resistance to transmissible subacute spongiform encephalopathies, characterized in that it comprises the embodiment of the previously described method.

The invention also proposes a method to screen an ovine population in relation to its sensitivity to transmissible subacute spongiform encephalopathies, characterized in that it comprises the embodiment of the previously described method.

This particularly allows breeders to set aside sensitive animals or only to retain resistant animals. These screening methods can especially be used under a reproduction plan.

Advantageously, the screening method comprises a step to compare the signal measured on the sample(s) to be analyzed, with resistant and/or sensitive sample(s).

The invention further proposes a kit to identify a genotype at position 171 of the ovine PrP, comprising:
a solid phase on which at least one capture antibody is immobilized, chosen in particular from among the antibodies SAF-15, SAF-31, SAF-32, SAF-33, SAF-34, SAF-35, SAF-37, 3B5, SAF-84, SHA-31, BAR-222, BAR-224, BAR-233, and 8G8;
a denaturing and reducing solution, and
at least one detection antibody, chosen in particular from among the labeled antibodies 12F10, BAR226, 11C6, and 2A11, and more particularly labeled 2A11.

The invention also proposes a kit for genotype identification at position 171 of the ovine PrP, characterized in that it comprises:
a solid phase on which at least one capture antibody is immobilized, chosen in particular from among the antibodies 12F10, BAR226, 11C6, and 2A11,
a denaturing and reducing solution, and
at least one detection antibody, chosen in particular from among the labeled antibodies SAF-15, SAF-31, SAF-32, SAF-33, SAF-34, SAF-35, SAF-37, 3B5, SAF-84, SHA-31, BAR-222, BAR-224, BAR-233, and 8G8.

The invention will be better understood and other characteristics and advantages thereof will become more clearly apparent on reading the following explanatory description given with reference to the figures in which:

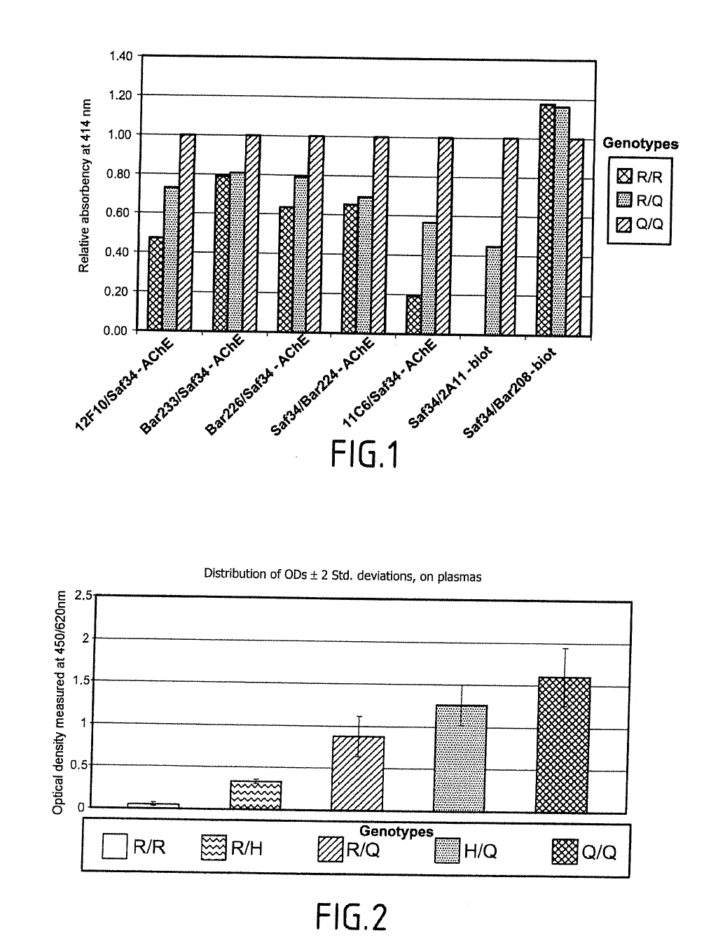

FIG. 1 illustrates the relative absorbencies, normalized with respect to genotype Q/Q, obtained with plasmas of ARR/ARR sheep, i.e. having the R/R genotype at position 171 of the PrP, or ARR/VRQ sheep, i.e having the R/Q genotype at position 171 of the PrP, or VRQ/VRQ sheep, i.e. having the Q/Q genotype at position 171 of the PrP.

These tests, of ELISA sandwich format, are conducted after denaturing and reducing the plasma as described in example 1. All the sandwiches involve the SAF-34 antibody (anti octa repeat) which indifferently recognizes all allotypes at position 171. Depending on cases, the SAF-34 antibody is used as capture antibody (SAF-34) or as detection antibody (SAF-34-ACNE, SAF-34 labeled with gymnote acetylcholinesterase);

FIG. 2 illustrates the distribution of optical densities (ODs) obtained with plasmas of sheep having the amino acids R/R, or R/H, or R/Q, or H/Q, or Q/Q at position 171 of the PrP. These tests were conducted using the SAF-34 antibody as capture antibody and the biotin-labeled 2A11 antibody as detection antibody.

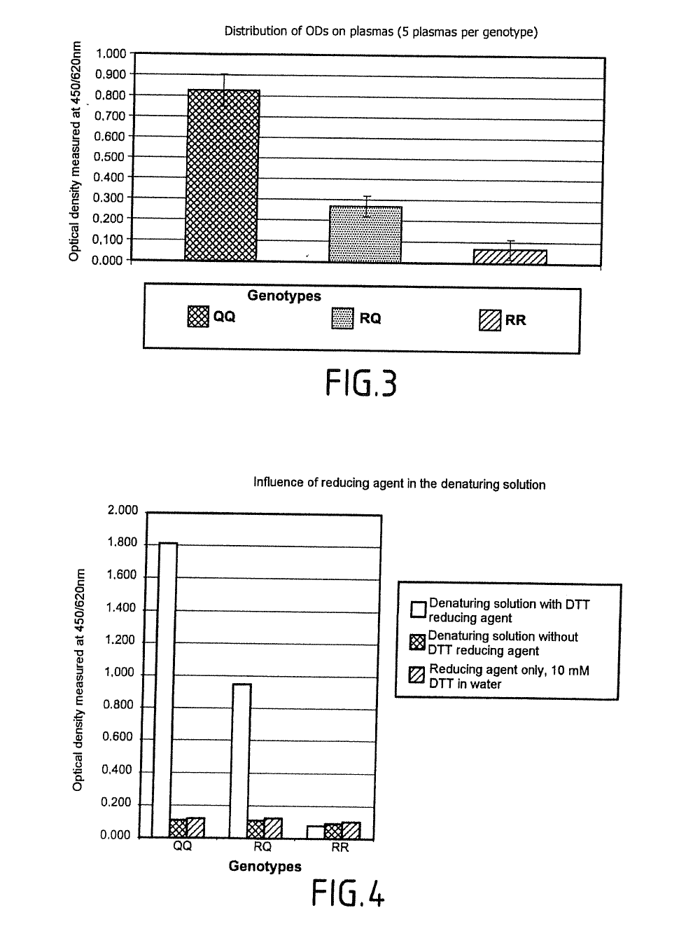

FIG. 3 illustrates the distribution of optical densities (ODs) obtained with plasmas of sheep having the amino acids Q/Q, or the amino acids R/Q, or the amino acids R/R at position 171. These tests were conducted using SAF-34 as capture antibody and the AChE-labeled 2A11 antibody as detection antibody,

FIG. 4 illustrates the influence of the presence or absence of a reducing agent which is dithiothreitol, and of the absence of a denaturing agent i.e. urea, in the composition of the denaturing solution. These tests were conducted using the biotin-labeled 2A11 antibody as detection antibody, and SAF-34 as the capture antibody.

FIG. 5 illustrates the distribution of optical densities (ODs) obtained with sera of sheep having the amino acids R/R, or R/H, or R/Q, or H/Q, or H/H, or Q/Q at position 171 of the PrP. These tests were conducted using antibody 3B5 as capture antibody, and biotin-labeled 2A11 as detection antibody with a DTT concentration of 20 mM.

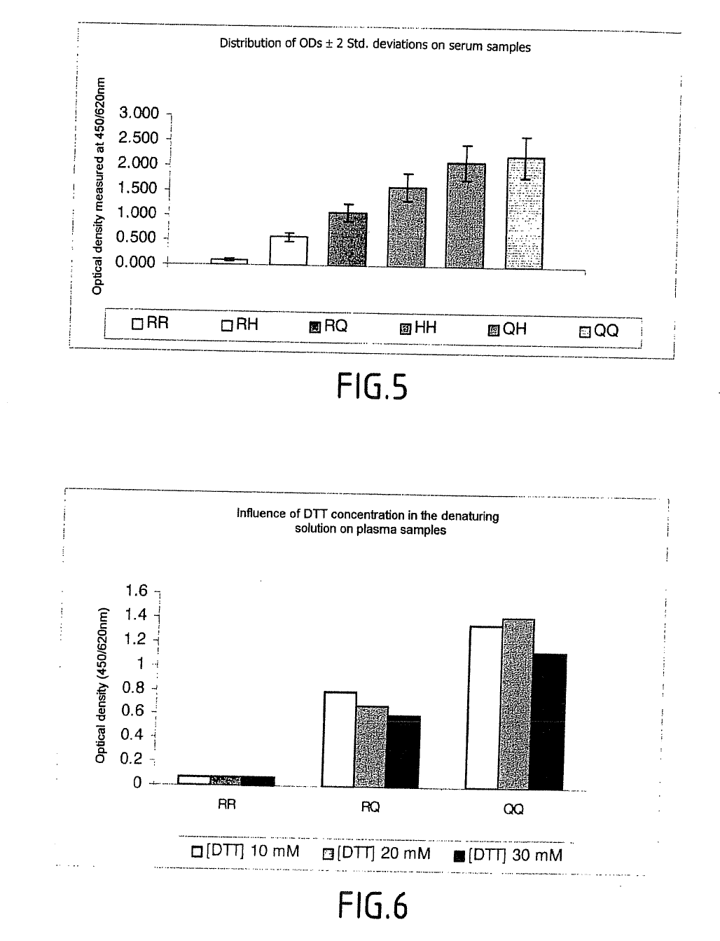

FIG. 6 illustrates the influence of DTT concentration in the denaturing solution on plasma samples. These tests were conducted using 3B5 as capture antibody and biotin-labelled 2A11 as detection antibody.

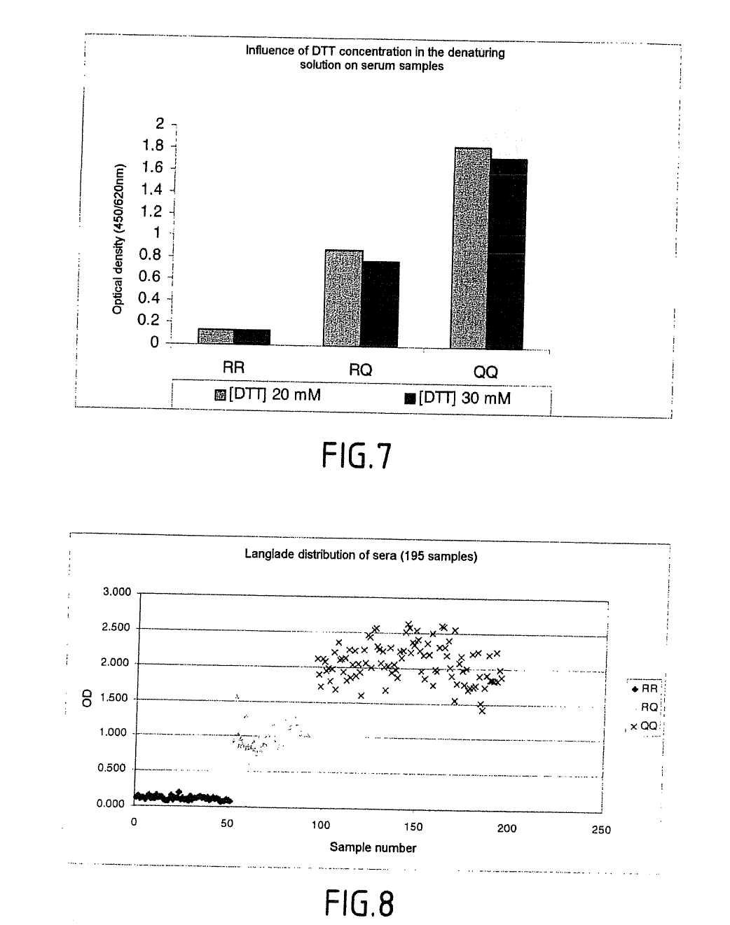

FIG. 7 illustrates the influence of DTT concentration in the denaturing solution on sera samples. These tests were conducted using antibody 3B5 as capture antibody and biotin-labeled 2A11 as detection antibody.

FIG. 8 illustrates the distribution of optical densities obtained in relation to different serum samples taken from sheep, which proved to correspond to different genotypes of the PrP. Tests were conducted using antibody 3B5 as capture antibody, and biotin-labeled 2A11 as detection antibody.

The object of the invention is a method for genotype identification at position 171 of the ovine PrP, in which an ovine biological fluid to be tested containing PrP is treated with a denaturing and reducing solution, the denatured and reduced PrP is immobilized, and the denatured and reduced PrP is contacted with at least one antibody specifically binding to the ovine PrP having a particular allelic form at position 171, and the possible presence of the PrP antibody is detected.

Within the scope of the invention, a “biological fluid” can in particular be blood, plasma, serum, urine, cerebrospinal fluid, saliva, milk etc. . . .

According to a preferred embodiment, this biological fluid is chosen from among blood, serum, plasma and milk. This biological fluid contains the PrP.

The term “antibody” refers to any whole antibody or to a functional fragment of an antibody comprising or consisting of at least one antigenic combination site, enabling said antibody to bind with at least one antigenic determinant of an antigenic compound. As examples of antibody fragments mention may be made of the fragments Fab, Fab′, F(ab′)₂ and the scFv chains (Single chain variable fragment), dsFv chains (Double-stranded variable fragment), etc. . . . These functional fragments can in particular be obtained by genetic engineering.

The production of monoclonal antibodies or of monospecific polyclonal serums which can be used under the invention lies within conventional techniques which are detailed further on.

The term “specifically”, when it refers to a specific recognition of, or to a specific binding with a particular allelic form at position 171 of the ovine PrP by an antibody, means that the antibody interacts preferably with the particular allelic form as compared with the other possible allelic forms, making it possible to differentiate and discriminate between possible allelic forms. Association constants of more than 10⁸ L·mol⁻¹ are preferable.

The antibodies used in the present invention are antibodies specifically directed against the prion protein, and on this account they are preferably monoclonal antibodies or monospecific polyclonal antibodies i.e. they do not recognize other proteins.

The monoclonal antibodies can be obtained using the conventional method of lymphocyte fusion and hybridoma culture described by Köhler and Milstein, (1975). Other methods to prepare monoclonal antibodies are also known (Harlow et al. (1988)). Monoclonal antibodies can be prepared by immunizing a mammal (e.g. mouse, rat, rabbit, even a human, etc. . . . ) and using the lymphocyte fusion technique leading to hybridomas (Köhler and Milstein, 1975).

There are alternative techniques to this usual method. It is possible for example to produce monoclonal antibodies by expressing a nucleic acid cloned from a hybridoma. It is also possible to produce antibodies using the <<phage display>> technique, by adding antibody cDNAs to vectors which are typically filamentous phages (e.g. fUSE5 for E. coli, Scott et al. (1990)). The latter form banks and have scFv fragments on their surface. Construction protocols for these antibody banks are described in Marks et al. (1991).

Polyclonal antibodies can be obtained from the serum of an animal immunized against an antigen of peptide type, following usual operating modes.

As a general rule it is possible to use a polypeptide for example as immunogen, particularly a recombined polypeptide, or an oligopeptide. Following conventional protocol, rabbits are immunized with the equivalent of 1 mg of peptide immunogen following the procedure described by Benoit et al. (1982). At four-week intervals, the animals are treated with injections of 200 μg of antigen and bled 10 to 14 days later. After the third injection, the capacity of the anti-serum to bind with the iodine-radiolabeled peptide antigen is evaluated, prepared using the chloramine-T method. It is then purified by ion-exchange chromatography on a carboxymethyl cellulose (CMC) column. The antibody molecules collected by elution are then adjusted to the desired concentration using methods well known to those skilled in the art e.g. using DEAE Sephadex to obtain the IgG fraction.

To increase the specificity of the polyclonal serum, the antibodies can be purified by immunoaffinity (or immunoadsorption) chromatography using titer peptides which were used for immunization and immobilized on a solid phase. The antiserum is contacted with said peptide immobilized on a solid phase for a sufficient time to cause the peptide to immuno-react with the antibody molecule to form a solid phase immunological complex.

As suitable antibody specifically binding with a particular allelic form at position 171 of the PrP, mention may be made of the antibodies 2A11, 11C6, BAR226, 12F10, V5 and V61.

However, as antibody specifically binding with the PrP having a particular form at position 171, preference is given to antibody 2A11, antibody 11C6, antibody BAR226 or antibody 12F10.

More particular preference is given to antibody 2A11. This antibody was produced following the protocol described in J. Brun et al; Neuroscience Research 48, 2004, 75-83, by immunizing PrP^{0/0} mice with recombinant bovine PrP. This antibody binds with the 171-179 sequence (QVYYRPVDQ) of bovine PrP and shows strong cross reactions with the PrP of most mammalian species which share the same epitope (sheep, goat, cat, rabbit, mouse, pig and hamster) but not with human PrP which has the sequence (QVYYRPMDE). It has also been shown that its affinity is strongly increased when the PrP is reduced and denatured, most probably on account of the proximity of the epitope with the first serine residue involved in a disulfide bridge of the PrP. Additionally, immunohistochemical analysis shows that immunoreactivity is greatly increased after treatment with proteinase K.

Also, it has been shown that the 2A11 antibody binds specifically with the allelic forms of sheep PrP carrying a residue of glutamine (Q) or histidine (H) at position 171, whereas no immunoreactivity is observed with the ARR allele.

In other words, the 2A11 antibody does not bind to the R residue. Therefore, the non-binding between the 2A11 antibody, used both as detection antibody and as capture antibody, with the PrP protein, a non-binding which translates as an optical density that is zero or close to zero, in the identification method of the invention, proves that the genotype at position 171 of the PrP contained in the biological fluid being tested is the R/R genotype, i.e. the genotype A₁₃₆R₁₅₄R₁₇₁/A₁₃₆R₁₅₄R₁₇₁.

Since, in addition, the 2A11 antibody binds in quantitatively different and preferable manner with the Q residue at position 171 of the Prp, as compared with the H residue, if any binding between the 2A11 antibody and the PrP is detected, it will be possible depending on the intensity of the signal obtained (translating as the OD) to discriminate between a biological fluid derived from a Q/Q homozygous sheep and a biological fluid derived from a Q/H heterozygous sheep or from a H/H homozygous sheep.

Similarly, heterozygous animals carrying a single R allele at position 171 (R/Q or R/H) will produce an even weaker signal since one half of the PrP contained in the sample is not recognized by the 2A11 antibody.

It is therefore indeed the existence of possible binding between the PrP of the sample and the specific antibody which is detected in the method of identification of the invention.

It has hence been possible to design a simple test of immunological type with which to evidence a distinct difference between animals, on the basis of the expressed genotype. According to a first embodiment, this test can be used to identify ARR/ARR animals. According to a second embodiment, this test can be used to identify heterozygous animals (R/(Q,H)) as compared with other genotypes (Q/Q, Q/H, H/H).

The method of the invention comprises the contacting of the biological fluid sample with a suitable quantity of a denaturing and reducing solution.

Advantageously, said solution comprises:

a) at least one denaturing agent chosen from among:
a surfactant chosen from the group consisting of:
   anionic surfactants, such as SDS (sodium dodecylsulfate), sarcosyl (lauroyl sarcosine), sodium cholate, sodium glycocholate, sodium deoxycholate, sodium taurocholate, sodium caprylate, sodium 1-decanesulfonate, sodium laurylsulfate and lithium laurylsulfate;
   zwitterionic surfactants such as SB 3-10 (decyl-sulfobetaine), SB 3-12 (dodecyl-sulfobetaine), SB 3-14 (tetradecyl-sulfobetaine), SB 3-16 (hexadecyl-sulfobetaine), SB 3-18 (octadecyl-sulfobetaine), CHAPS and CHAPSO and deoxy CHAPS;
   non-ionic surfactants, such as Triton X-100, Triton X-114, Tween 20, Tween 80, Brij 35 (polyoxyethylene 23 laurylether), nonidet P-40, n-decyl-beta-D-glucopyranoside, n-dodecyl-beta-D-glucopyranoside, n-octyl-beta-D-glucopyranoside, n-octyl-alpha-D-glucopyranoside;
   mixtures of these surfactants, and/or
a chaotropic agent, and

b) at least one reducing agent.

By reducing agent is meant an agent capable of cleaving the disulfide bridge of the PrP protein.

Among the reducing agents which may be used in the present invention, particular mention may be made of DTT (dithiothreitol), TCEP (Tris(2-carboxyethyl)phosphine hydrochloride), DTE (dithio erythritol), Beta mercaptoethanol, 2-mercaptorthylamine or a mixture thereof.

The reducing agent is generally present in the mixture consisting of the sample to be treated and the denaturing and reducing solution at a content of between 2.5 mM and 100 mM, in particular from 5 mM to 50 mM, and more particularly a content ranging from 5 mM to 30 mM.

The chaotropic agent is chosen from among urea, guanidine, guanidine hydrochloride, guanidine thiocyanate or one of their mixtures.

If chaotropic agents are present, their concentration is generally equal to or more than 1 M, preferably more than 3 M.

The preferred chaotropic agent is urea, preferably at a concentration of 8 M.

The mixture consisting of the sample to be treated and the denaturing and reducing solution is generally heated to a temperature of between 37° C. and 100° C., in particular from 50° C. to 70° C., and more particularly at around 60° C. for 5 minutes to one hour, in particular between 7 and 20 minutes and more particularly for approximately 10 minutes.

These conditions are chosen to meet the contradictory requirements of sufficient deterioration of the PrP for the epitope formed by the amino acid at position 171 to be unmasked, and non-excessive deterioration of the PrP so that the epitope is still able to be recognized by the antibody. Additionally, this treatment must not affect subsequent fixing of the antibody.

According to a particular embodiment, said solution comprises a mixture of surfactants, notably a mixture of ionic surfactants, in particular a mixture of anionic surfactants, and more particularly a mixture of SDS and sarcosyl.

The concentration of surfactants in the mixture consisting of the sample to be treated and the denaturing and reducing solution is generally equal to or more than 0.5 wt. % relative to the total volume of the mixture, in particular equal to or more than 2 wt. % relative to the total volume of the mixture.

Therefore the denaturing and reducing solution comprises at least one denaturing agent which may be a surfactant and/or a chaotropic agent and at least one reducing agent.

Treatment of the PrP with this solution that is both denaturing and reducing is indispensable for the embodiment of the method of the invention, as will be shown under example 4.

Generally, the solution containing the antibody specific to the amino acid at position 171 is left in contact with the solid phase on which the PrP is immobilized for at least 10 nm, in particular for approximately one hour, at a temperature possibly ranging from 4 to 50° C., and in particular at 37° C.

Evidently, the method to detect labeling on the solid phase depends on the labeler used. These are methods well known to those skilled in the art. Mention may be made of biotin labeling for example. For example, in this case, the possible presence of biotin immobilized on the support is developed by adding a solution of streptavidine-peroxidase conjugate which is left to react for around 10 to 30 minutes at a temperature of approximately 37° C. Finally, the presence of peroxidase activity bound to the support is developed through the addition of a chromogenic substrate e.g. tetramethylbenzidine, which is left to react for around 30 minutes at 20° C. Finally, absorbency is measured at 450 and 620 nm.

Regarding the immobilization of the denatured, reduced prion protein, this may entail direct immobilization on the solid phase or indirect immobilization.

The solid phase may be a microtiter plate, beads, tubes, in polymer in particular in polystyrene, polyethylene or latex.

Preferably the solid phase is a microtiter plate in polystyrene.

With respect to immobilization of the PrP directly on the solid phase, the prion protein which fixes to the plate, like all proteins, must be purified (denatured) to remove as many non-desired proteins as possible from the sample, before immobilizing the PrP protein itself.

Additionally, the prion protein must be reduced.

Next, the detection antibody which is directly or indirectly labeled is caused to react.

Regarding indirect immobilization of the PrP on the solid phase, immobilization can be conducted using an antibody or another specific ligand which is not an antibody. Thus, indirect immobilization can be carried out via an antibody, called a capture antibody, which is capable of retaining the PrP by affinity binding, or via an antibody which specifically binds to a particular form of the allele at position 171, or via a ligand which is not an antibody. This ligand can be a molecule such as plasminogen, avidine, streptavidine, glycose aminoglycans, hesperidine, porphyrines, streptamycine and tetracycline.

If the PrP is immobilized via a ligand or an antibody capable of retaining the PrP by affinity binding, the immobilized PrP protein is caused to react with an antibody which specifically binds to a particular form of the 171 allele, which is labeled directly or indirectly.

On the other hand, if the Prp is immobilized on the solid phase via an antibody specifically binding to a particular form of the allele at position 171, the immobilized PrP protein is then caused to react with an antibody capable of retaining it by affinity binding.

In this case, it is this antibody which is labeled directly or indirectly.

Then, in all cases, detection is made of the labeled antibody.

The method of the invention is advantageously implemented in the form of an immunoassay of sandwich type, particularly of ELISA type. In said case, it is suitable to use both a so-called capture antibody and a so-called detection antibody able to bind simultaneously with the purified, reduced PrP protein.

By capture antibody is meant an antibody, or part of antibody, preferably fixed to a solid phase which is capable of retaining an antigen present in a biological sample, either by simple affinity binding or via affinity binding by recognition of a particular epitopic site.

When the antigen is immobilized by a capture antibody, the detection antibody must be capable of binding itself to an epitopic site that is still accessible and different from the site recognized by the capture antibody.

The term <<labeled>> refers both to direct labeling (via enzymes, radioisotropes, fluorochromes, luminescent compounds, etc.) and to indirect labeling e.g. via antibodies which themselves are labeled directly or using reagents of a labeled <<affinity pair>> such as, but not exclusively, the biotin-labeled avidine pair, etc.

Therefore a particular subject of the invention is a method for genotype identification at position 171 of the PrP, comprising the steps consisting of:
treating a sample of biological fluid to be tested, so as to denature and reduce the PrP contained in said sample,
contacting said treated sample with a solid phase on which a so-called capture antibody is immobilized,
washing the solid phase obtained in the preceding step,
contacting said washed solid phase with a solution of labeled antibody called detection antibody,
washing the solid phase obtained in the preceding step, then
detecting the possible presence of labeling on the solid phase,

one of the antibodies specifically recognizing the PrP, irrespective of the allele variant at position 171 of the PrP, the other antibody binding specifically to the ovine PrP having a particular allele variant at position 171.

As antibody specifically recognizing the PrP irrespective of the allele variant at position 171 of the PrP, particular mention may be made of antibodies capable of specifically recognizing the octapeptide repeat structure, such as those described in patent application WO 01/35104. Amongst these antibodies, particular mention may be made of those chosen from the group consisting of antibodies SAF-15, SAF-31, SAF-32, SAF-33, SAF-34, SAF-35, SAF-37 described in application WO01/35104.

Mention may also be made of antibody 3B5 described in Krasemann et al. Krasemann, S., Groschup, M. H., Harmeyer, S., Hunsmann, G. and Bodemer, W. (1996a) Generation of monoclonal antibodies against human prion proteins in PrP^{0/0} mice. Molecular Medecine, 2, 725-734.

Additionally the article by Féraudet et al can be cited (Screening of 145 Anti-PrP Monoclonal Antibodies for Their Capacity to Inhibit PrPSc Replication in Infected Cells, the Journal of Biological Chemistry, Vol. 280, No. 12, Issue of March 25, pp. 11247-11258, 2005). Particular preference is given to the antibody SAF-34 or the antibody 3B5.

As antibody specifically recognizing a particular allele variant of the PrP at position 171, the antibody 2A11 is cited. Also cited are the antibody 12F10 described in Krasemann, S., Jurgens T. and Bodemer, W. (1999) Generation of monoclonal antibodies against prion proteins, an unconventional nucleic acid based immunization strategy. Journal of Biotechnology, 73, 119-129, and the antibody 11C6 described in Krasemann, S., Groschup, M. H., Hunsmann, G. and Bodemer, W. (1996b) Induction of antibodies against human prion proteins (PrP) by DNA-mediated immunization of PrP^{0/0} mice. J. Immunol. Methods, 199, 109-118.

Mention is also made of the V5 and V61 antibodies described in Moudjou et al, Journal of Virology, September 2004, page 9270-9276.

It is evidently within the reach of those skilled in the art to produce or procure monoclonal antibodies, having similar or identical epitopic specificity to that described for the foregoing antibodies, and which are suitable for the embodiment of the present invention.

The following figures and examples illustrate the invention without limiting the scope thereof.

### EXAMPLES

### Material

### Obtaining and Characterizing Monoclonal Antibodies Specific to the Octapeptide Repeat Structure (SAF).

The antibodies SAF-15, 31, 32, 33, 34, 35 and 37, specific to the octapeptide repeat structure were prepared as described in patent application WO 01/35104.

Synthesis and Labeling of the 79-92 Peptide of Human PrP

A peptide representing the PrP octapeptide repeat structure, e.g. structure G-G-W-G-Q-P-H-G-G-G-W-G-Q-G-(NH₂), corresponding to sequence 79-92 of the human PrP, was synthesized using an automatic synthesizer (Milligen 9050, Waters, Milford, Mich.). The peptide was coupled covalently with gymnote acetylcholinesterase (ACNE) via the hetero-bifunctional reagent succinimidyl 4-(N-maleimidométhyl) cyclohexane-1-carboxylate (SMCC, Calbiochem, France), as described previously for other peptides or proteins (McLaughlin et al, 1987, Grassi et al, 1989). This method involves the reaction of a thiol group, added to the peptide, with the maleimide function fixed to AChE by reaction with SMCC. The thiol group was added to the peptide by reaction with N-succinimidyl S-acétylthioacétate (SATA) as described previously (McLaughlin et al, 1987). Coupling was obtained by causing the AChE-SMCC to react with excess thiolated peptide.

Immunization and Preparation of Monoclonal Antibodies

A preparation of “Scrapie-associated fibrils” (SAFs, PrPres preparation) was obtained from infected hamster brains (scrapie strain 263 K) as described previously (Lasmezas et al, 1997). This preparation was inactivated by treating with formic acid before immunization of the mice. Knock-out mice for the PrP gene (PrP^{0/0} mice) were immunized with these SAF preparations, and hybridoma cells were prepared as described previously (Grassi et al, 1988, 1989). Screening of the culture supernatants was conducted as described below. 57 hybridomas were able to be identified and stabilized: they were called SAF-1 to SAF-90. All these antibodies proved to recognize the SAFs immobilized on microtiter plates, while a minority thereof showed the capacity to recognize peptide-AchE conjugates. Amongst the latter, seven distinctly recognize the octapeptide repeat structure (reaction with the 79-92 peptide coupled with AChE); they are the antibodies SAF-15, SAF-31, SAF-32, SAF-33, SAF-34, SAF-35 and SAF-37. After cloning and expansion in ascitis liquid form, the monoclonal antibodies were purified by affinity chromatography on a column of Protein A Sepharose and stored at −20° C. until use. The isotype of the antibodies was determined by radial immunodiffusion following Ouchterlony's technique.

Screening Hybridoma Culture Supernatants

The presence of PrP-specific antibodies in the supernatants of hybridoma culture was evidenced in two manners, testing either their capacity to bind peptide-AChE conjugates (in particular peptide 79-92-AChE) or hamster SAFs. In the first case, screening was conducted in plates containing a goat anti-mouse IgG antibody immobilized as described previously (Créminont et al, 1993, Frobert et al, 1991). To summarize, 100 μl of culture supernatants and 100 μl of Peptide-AChE conjugate were left to react overnight at 4° C. in plates containing immobilized goat anti-mouse IgG antibodies. After washing the plates, 200 μl of Ellman reagent (Ellman et al, 1961) were added to the wells to detect the presence of AChE fixed to the solid phase. In the second case, plates containing an immobilized SAF preparation were prepared by causing 50 μl of a 2 μg/ml solution to react in a 0.05 M phosphate buffer, pH 7.4, overnight at ambient temperature. After washing, the plates were saturated with EIA buffer (100 mM phosphate buffer, pH7.4 containing 150 mM NaCl, 0.1% bovine serum albumin (BSA) and 0.01% sodium azide) overnight at 4° C., and were held at this temperature until their use. The binding of the monoclonal antibodies to the immobilized SAFs was evidenced using AchE-labelled goat anti-mouse IgG antibodies, as described previously (Negroni et al, 1998).

Labeling Monoclonal Antibodies with AChE.

Monoclonal antibodies labeled with AchE are prepared by coupling reduced Fab′ fragments with the tetrameric form of the enzyme via a heterobifunctional reagent, namely succinimidyl 4-(N-maleimidomethyl)cyclohexane-1-carboxylate (SMCC, Calbiochem, France), as described previously (Grassi et al, 1989). This method entails the reaction of a thiol group carried by the reduced Fab′ fragment, with the maleimide function which has been fixed to AChE by reaction with SMCC.

### Obtaining the Monoclonal Antibody 3B5, 11C6 and 12F10

The monoclonal antibodies 3B5, 11C6 and 12F10 were obtained after immunizing knock-out mice for the PrP with recombinant human PrP and were screened following different techniques as described in Krasemann et al.

3B5 is an antibody directed against recombinant human PrP and recognizes peptide 79-92.

11C6 is an antibody which recognizes a non-identified conformational epitope.

12F10 is an antibody directed against recombinant human PrP and recognizes peptide 142-160.

### Obtaining the Monoclonal Antibody Bar226

The antibody Bar-226 was obtained by immunizing knock-out mice for the PrP gene with recombinant PrP (ARQ allotype) as described in C. Feraudet, N. Morel, S. Simon, H. Volland, Y. Frobert, C. Creminon, D. Vilette, S. Lehmann, J. Grassi (2005) Screening of 145 anti PrP Monoclonal antibodies for their capacity to inhibit PrPSc Replication in Infected Cells J. Biol. Chem., 280, 11247-11258.

### Obtaining the Monoclonal Antibody 2A11

The monoclonal antibody 2A11 is prepared following the protocol described in Brun et al; J. Neuroscience Research 48, 2004, 75-83.

### Conjugate: Monoclonal Antibody 2A11-Biotin

A conjugate of the monoclonal antibody 2A11 labeled with biotin is prepared following the protocol described by Greg T. Hermanson in 1996.

Conjugate: Streptavidine Labeled with Peroxidase

A conjugate of streptavidine labeled with peroxidase is prepared following the protocol described by Greg T. Hermanson in 1996.

### Biological Fluids

The biological fluids used were serums or plasmas from sheep of races Cheviot, Romanov, Casserarde and Manech.

### Ambient Temperature

Ambient temperature is defined as a temperature between 18° C. and 30° C. inclusive.

### Example 1

### Conducting of the Test with Different Pairs of Antibodies

Plasma samples were taken from sheep having the genotype R/R, or R/Q or Q/Q at position 171.

65 μl of each sheep plasma sample were mixed with 65 μl of denaturing and reducing solution consisting of 8M urea and 10 mM DTT. The mixture was then heated for 30 minutes at 50° C.

100 μl of the mixture obtained are deposited in a well of a microtiter plate containing a capture antibody which is either antibody 12F10, or antibody BAR233, or antibody BAR226, or antibody SAF-34, or antibody 11C6.

Three washings of the wells are then made with a washing solution containing a 10⁻² M phosphate buffer, pH 7.4 and 0.05% Tween 20.

After washing, 100 μl of detection antibody are deposited per well, which is either the AChE-labeled antibody SAF-34 if the capture antibody is the antibody 12F10 or BAR233 or BAR226 or 11C6, or the antibody BAR224 labeled with AChE, or the antibody 2A11 labeled with biotin, or the antibody BAR208 labeled with biotin, with 3 μg of antibody per liter relative to the final mixture thus obtained.

The wells are left to incubate one hour at 37° C.

Next, five washings are conducted with the washing solution containing 10⁻² M phosphate buffer, pH 7.4 and 0.05 wt. % Tween 20 relative to the total weight of the washing solution.

When the labeled antibodies are biotin-labeled antibodies 2A11 and Bar208, the presence of the labeled antibody on the solid phase is developed through the addition of a streptavidine-peroxidase conjugate at 0.2 μg/ml of the final mixture. In this case, they are left to incubate 30 minutes at 37° C. Then, 5 washings are carried out with a washing solution containing 10⁻² M phosphate buffer, pH 7.4 and 0.05 wt. % Tween 20 relative to the total weight of the washing solution. 100 μl of the peroxidase substrate and 2 ml of tetramethylbenzidine solution (TMB) as chromogen are deposited in each well. They are left to incubate 30 minutes at ambient temperature in darkness. 100 μl of 1N sulfuric acid solution, as stop solution, are deposited in each well. Absorbency is measured at 450 nm and 620 nm.

For the AchE-labeled antibodies, after adding 200 μl of Ellman reagent (Ellman et al., 1961) to the wells, the presence of AChE fixed to the solid phase is detected by measuring absorbency at 414 nm.

The results obtained are shown FIG. 1.

It can clearly be seen from FIG. 1 that the pairs of capture antibodies/detection antibodies 12F10/SAF-34-AChE, BAR226/SAF-34-AChE, 11C6/SAF-34-AChE, SAF-34/2A11-biotin allow a distinct differentiation to be made between the animals having genotype R/R at position 171 from the animals having the genotype R/Q and animals having the genotype Q/Q at this same position.

It is also ascertained that the other pairs of capture antibodies/detection antibodies do not allow discrimination to be made between the different genotypes at position 171 of the prion protein PrP.

It can also be clearly seen FIG. 1 that the SAF-34 antibody, which is an antibody which does not bind to a specific allelic form at position 171, can be used both as capture antibody and as detection antibody. When used as detection antibody it is labeled with AChE.

It can also be clearly seen from FIG. 1 that the pair allowing best discriminatory identification of animals with genotype R/R from animals having genotype R/Q and animals having genotype Q/Q at position 171 is the biotin-labeled SAF-34/2A11 pair.

Indeed the antibody 2A11 does not at all bind to the R allelic form at position 171 of the PrP.

### Example 2

### Conducting of the Test on Plasma Samples

Plasma samples were taken from sheep having the genotype R/R or R/H or R/Q or H/Q or Q/Q, at position 171 of the PrP.

75 μl of each sheep plasma sample were mixed with 75 μl of denaturing and reducing solution consisting of 2 wt. % sarcosyl relative to the total volume of the denaturing and reducing solution, 2 wt. % dodecylsulfate relative to the total volume of denaturing and reducing solution, and 10 mM dithiothreitol.

The mixture is then heated for 10 minutes at 60° C.

100 μl of the mixture obtained are deposited in a well of a microtiter plate containing a capture antibody which is the SAF-34 antibody.

Then, three washings of the wells are conducted with a washing solution containing 0.01 M Tris, 0.3 M NaCl, pH 7.4, and 0.1 wt. % Tween 20, relative to the total weight of the washing solution.

The washing solution is buffered to neutral pH or slightly alkaline and contains a neutral/non-ionic detergent in low concentration (0.01% Tween 20).

After washing, 100 μl of detection antibody are deposited per well, the antibody being the biotin conjugate 2A11 antibody, present in a concentration of 3 μg antibody per liter relative to the total weight of the final mixture thus obtained.

The wells are left to incubate one hour at 37° C.

Then three washings are carried out with the washing solution previously described.

The presence of the labeled antibody on the solid phase is developed through the addition of the streptavidine-peroxidase conjugate described under the paragraph “Material”, at 0.2 μg/ml of final mixture.

The wells are left to incubate 30 minutes at 37° C.

Five washings are carried out with the washing solution previously described.

100 μl of the peroxidase substrate and 2 ml tetramethylbenzidine solution (TMB), as chromogen, are deposited in each well.

The wells are left to incubate 30 minutes at ambient temperature in darkness.

100 μl of 1N sulfuric acid solution, as stop solution, are deposited in each well.

Absorbency (optical density) is measured at 450 and 620 nm.

The results obtained are given FIG. 2.

FIG. 2 shows the distribution of the optical densities obtained with the plasmas of each category of sheep genotypes.

As can be seen FIG. 2, the pair SAF-34 antibody/biotin-labeled 2A11 allows excellent discrimination to be achieved between the different sheep genotypes.

### Example 3

### Conducting the Test on Plasma Samples Under Different Conditions to Example 2

In this example, the denaturing agent is a chaotropic agent, more precisely 8 M urea.

Plasma samples were taken from sheep having the genotype R/R or R/Q or Q/Q at position 171 of the PrP.

65 μl of each sample of sheep plasma were mixed with 65 μl of denaturing and reducing solution consisting of 8 M urea and 10 mM dithiothreitol.

The mixture is then heated for 10 minutes at 60° C.

Next, 130 μl of EIA buffer (Enzyme Immuno Assay) are added containing a 1 M potassium phosphate buffer, pH 7.4, 0.15 M NaCl, and 0.1 wt. % bovine serum albumin (BSA) relative to the total volume of EIA buffer.

100 μl of the mixture obtained are deposited in a well of a microtiter plate containing a capture antibody which is the SAF-34 antibody.

The mixture is left to incubate one hour at 20° C.

Three washings of the wells are then carried out with a washing solution containing 0.01 M Tris, 0.3 M NaCl, pH 7.4, and 0.1 wt. % Tween 20 relative to the total volume of the washing solution.

After washing, 100 μl of detection antibody are deposited per well, the antibody being biotin-labelled 2A11, the 2A11 antibody-biotin conjugate having a concentration of 0.5 μg/ml relative to the final mixture thus obtained.

The wells are left to incubate one hour at +4° C.

Three washings are then carried out with the washing solution previously described.

After washing, 100 μl of streptavidine-peroxidase conjugate are deposited per well, as described under the paragraph <<Material>>, at a concentration of 0.1 μg/ml in the final solution.

The wells are left to incubate 30 minutes at +20° C.

Five washings are carried out with the washing solution previously described.

100 μl of peroxidase substrate and 2 ml tetramethylbenzidine solution (TMB) as chromogen are deposited in each well.

The wells are left to incubate 30 minutes at ambient temperature in darkness.

100 μl of 1 N sulfuric acid solution, as stop solution, are deposited in each well.

Absorbency is measured at 450 and 620 nm.

The results obtained are given FIG. 3.

By comparing FIGS. 2 and 3, it is found that the antibody pair SAF-34/2A11 allows excellent discrimination to be achieved between the different sheep genotypes, irrespective of the composition of the denaturing and reducing solution.

### Example 4

### Study on the Influence of the Presence of the Denaturing Agent/Reducing Agent Association

The purpose of this example was to evaluate the importance of having a reducing agent and a denaturing agent in the denaturing and reducing solution used.

To this end, plasmas from sheep having genotype Q/Q or R/R at position 171 of the PrP were tested following the protocol in example 3, i.e. with a solution containing 8 M urea as denaturing agent and 10 mM DTT as reducing agent.

The same sheep plasmas were tested with a solution solely containing a denaturing agent i.e. 8 M urea.

These same samples were also treated with a solution solely containing a reducing agent i.e. 10 mM DTT.

The optical density of each of these plasmas was measured at 450-620 nm.

The results obtained are illustrated FIG. 4 in which the white columns represent the optical densities obtained with the plasmas treated with a solution containing a denaturing agent and a reducing agent, the dotted columns represent the densities obtained with samples of biological fluid treated with a solution only containing a denaturing agent and not containing any reducing agent, and the cross-hatched columns represent the optical densities obtained with the samples treated with a solution only containing a reducing agent and not containing any denaturing agent.

As can be seen FIG. 4, the combined presence of a reducing agent and a denaturing agent (surfactant and/or chaotropic agent) is absolutely necessary to discriminate between the different genotypes.

It is to be noted that since the antibody 2A11 does not bind to the R allelic form at position 171 of the PrP, the results obtained do not show any difference between the tests conducted with a denaturing solution whether or not they contain a reducing agent.

The same tests as in examples 1 to 4 were conducted on sheep sera. Similar results were obtained.

Once the genotype at position 171 of the protein been identified, it is possible to select sheep intended for reproduction.

Preferably, the selected sheep are those having genotype R/R which are the most resistant to scrapie.

Therefore it will be possible, in accordance with plans, to select male and female sheep having this genotype so that only sheep of genotype R/R are produced, or to select solely male sheep of genotype R/R and allow them to reproduce with females of all genotypes.

Advantageously, the method of the invention can be implemented by means of kits.

In a first embodiment, the kit of invention comprises:
a solid phase on which at least one capture antibody is immobilized chosen from among the antibodies SAF-15, SAF-31, SAF-32, SAF-33, SAF-34, SAF-35, SAF-37, 3B5, SAF-84, SHA-31, BAR-222, BAR-224, BAR-233, and 8G8,
a denaturing and reducing solution, and
at least one detection antibody, chosen in particular from among the labeled antibodies 12F10, BAR226, 11C6, and 2A11, and more particularly labeled 2A11.

In a second embodiment, the kit of the invention comprises:
a solid phase on which at least one capture antibody is immobilized chosen from among the antibodies 12F10, BAR-226, 11C6, and 2A11,
a denaturing and reducing solution, and
at least one detection antibody chosen from among the antibodies SAF-15, SAF-31, SAF-32, SAF-33, SAF-34, SAF-35, SAF-37, 3B5, SAF-84, SHA-31, BAR-222, BAR-224, BAR-233 and 8G8, that are labeled.

Preferably, in the kits of the invention the solid phase is a microtiter plate, preferably in polystyrene.

Preferably, in the first embodiment of the kit of the invention, the capture antibody is the SAF-34 antibody or 3B5 antibody, and the detection antibody is the 2A11 antibody.

Also preferably, in the kits of the invention the denaturing solution contains sodium dodecylsulfate, dithiothreitol and sarcosyl.

### Example 5

### Conducting the Test on Sera

A study was made of the distribution of optical densities (OD) obtained on serums of sheep having the amino acids R/R, or R/H, or R/Q, or H/Q, H/H and Q/Q at position 171 of the PrP, following a protocol similar to the one described in example 2, except that the sample studied is a serum, the DTT concentration is 20 mM, the capture antibody is 3B5, the detection antibody which is 2A11 is used at 2.2 μg/L of antibody relative to the total weight of the final mixture thus obtained, and the presence of the labeled antibody on the solid phase is developed through the addition of the streptavidine/peroxidase conjugate described under the paragraph “Material” at a concentration of 0.37 μg/mL of final mixture.

The results obtained are shown FIG. 5.

A good discrimination is observed between the different sheep genotypes.

### Example 6

### Study on the Influence of the Concentration of Reducing Agent on Plasma Samples

Plasma samples were taken from sheep of genotype R/R or R/Q or Q/Q, at position 171 of the PrP.

75 μl of each sheep plasma sample were mixed with 75 μl of denaturing and reducing solution consisting of 2 wt. % sarcosyl, relative to the total volume of denaturing and reducing solution, 2 wt. % dodecylsulfate relative to the total volume of denaturing and reducing solution, and 10 mM, 20 mM or 30 mM dithiothreitol.

The mixture is then heated for 10 minutes at 60° C.

100 μl of the mixture obtained are deposited in a well of a microtiter plate containing a capture antibody which is the 3B5 antibody.

Three washings of the wells are then carried out with a washing solution containing 0.01M Tris, 0.3 M NaCl, pH 7.4, and 0.1 wt. % Tween 20 relative to the total weight of the washing solution.

The washing solution is buffered to neutral pH or slightly alkaline, and contains a low concentration neutral/non-ionic detergent (Tween 20 at 0.01%).

After washing, 100 μl of detection antibody are deposited per well, which is the conjugate biotin 2A11 antibody, which is present at a concentration of 3 μg antibody per liter relative to the total weight of the final mixture thus obtained.

The wells are left to incubate one hour at 37° C.

Three washings are then carried out with the previously described washing solution.

The presence of the labeled antibody on the solid phase is developed through the addition of the streptavidine-peroxidase conjugate described under the paragraph “Material”, at 0.4 μg/ml of final mixture.

This is left to incubate 30 minutes at 37° C.

Next, five washings are conducted with the previously described washing solution.

100 μl of the perioxidase substrate and 2 ml tetramethylbenzidine solution (TMB), as chromogen, are deposited in each well.

The wells are left to incubate 30 minutes at ambient temperature in darkness.

100 μl of 1N sulfuric acid solution, as stop solution, are deposited in each well.

Absorbency (optical density) is measured at 450 and 620 nm.

The results obtained are shown FIG. 6.

It is observed that the variation in concentration of reducing agent has no significant impact on the discrimination between populations.

### Example 7

### Study on the Influence of Reducing Agent Concentration on Serums

Serum samples were taken from sheep of genotype R/R or R/Q or Q/Q, at position 171 of the PrP.

75 μl of each sheep sample were mixed with 75 μl of denaturing and reducing solution consisting of 2 wt. % sarcosyl, relative to the total volume of denaturing and reducing solution, 2 wt. % dodecylsulfate relative to the total volume of denaturing and reducing solution, and 20 mM or 30 mM dithiothreitol.

The mixture is then heated for 10 minutes at 60° C.

100 μl of the mixture obtained are deposited in a well of a microtiter plate containing a capture antibody which is the antibody 3B5.

Three washings of the wells are then carried out with a washing solution containing 0.01 M Tris, 0.3 M NaCl, pH 7.4, and 0.1 wt. % Tween 20 relative to the total weight of the washing solution.

The washing solution is buffered to neutral pH or slightly alkaline and contains a low concentration neutral/non-ionic detergent (0.01% Tween 20).

After washing, 100 μl of detection antibody are deposited per well, which is the biotin conjugate 2A11 antibody and is present at a concentration of 2 μg antibody per liter relative to the total weight of the final mixture thus obtained.

The wells are left to incubate one hour at 37° C.

Three washings are then carried out with the previously described washing solution.

The presence of the labeled antibody on the solid phase is developed through the addition of the streptavidine-peroxidase conjugate described under the paragraph “Material”, at 0.33 μg/ml of final mixture.

This is followed by incubation for 30 minutes at 37° C.

Five washings are then carried out with the previously descried washing solution.

100 μl of the peroxidase substrate and 2 ml tetramethylbenzidine solution (TMB), as chromogen, are deposited in each well.

The wells are left to incubate 30 minutes at ambient temperature in darkness.

100 μl of 1N sulfuric acid solution, as stop solution, are deposited in each well.

Absorbency (optical density) is measured at 450 and 620 nm.

The results obtained are given in FIG. 7.

It is observed that the variation in concentration of reducing agent has no significant impact on the discrimination between populations.

### Example 8

### Blind Test

195 serum samples are taken from different sheep.

The protocol described in example 5 is followed on the 195 collected samples.

The protocol described in example 5 is also followed using serums whose genotypes R/R, R/Q and Q/Q are known (controls)

The results obtained are illustrated in FIG. 8.

It is observed that the genotype of the tested sample can be identified most advantageously.

1. A method for identifying the genotype at position 171 of the ovine PrP, the method comprising the steps of:
a) treating a sample of ovine biological fluid to be tested containing the PrP, with a denaturing and reducing solution,
b) immobilizing, optionally via a ligand, the denatured and reduced PrP on a solid phase,
c) contacting the denatured, reduced and immobilized PrP with at least one detection antibody, and
d) detecting the possible presence of said at least one detection antibody,
wherein one of the ligand or of said at least one detection antibody specifically binds to the PrP having a particular allelic form at position 171.

2. The method according to claim 1, wherein the denaturing solution and reducing solution further comprises:
a) at least one denaturing agent selected from the group consisting of:
a surfactant selected from the group consisting of:
anionic surfactants, zwitterionic surfactants, nonionic surfactants, mixtures thereof, and
a chaotropic agent; and
b) at least one reducing agent.

3. The method according to claim 2, wherein the chaotropic agent is selected from the group consisting of urea, guanidine, guanidine hydrochloride and guanidine thiocyanate or one of their mixtures.

4-26. (canceled)

27. The method according to claim 2, wherein the surfactant is selected from the group consisting of SDS (sodium dodecylsulfate), sarcosyl (lauroyl sarcosine), sodium cholate, sodium glycocholate, sodium deoxycholate, sodium taurocholate, sodium caprylate, sodium 1-decanesulfonate, sodium laurylsulfate, lithium laurylsulfate, SB 3-10 (decyl-sulfobetaine), SB 3-12 (dodecyl-sulfobetaine), SB 3-14 (tetradecyl-sulfobetaine), SB 3-16 (hexadecyl-sulfobetaine), SB 3-18 (octadecyl-sulfobetaine), CHAPS, CHAPSO, deoxy CHAPS, Triton X-100, Triton X-114, Tween 20, Tween 80, Brij 35 (polyoxyethylene 23 laurylether), nonidet P-40, n-decyl-beta-D-glucopyranoside, n-dodecyl-beta-D-glucopyranoside, n-octyl-beta-D-glucopyranoside, and n-octyl-alpha-D-glucopyranoside.

28. The method according to claim 1, wherein said denaturing and reducing solution further comprises a mixture of ionic surfactant agents.

29. The method according to claim 1, wherein the denaturing and reducing solution contains at least 0.5 wt. % surfactant agent relative to the total volume of the mixture comprising the sample to be treated, the denaturing solution, and the reducing solution.

30. The method according to claim 2, wherein said at least one reducing agent is selected from the group consisting of DTT (dithiothreitol), TCEP (Tris(2-carboxyethyl)phosphine) hydrochloride, DTE (dithio erythritol), beta mercaptoethanol, 2-mercaptoethylamine, and one of their mixtures.

31. The method according to claim 2, wherein the concentration of reducing agent is comprises from about 2.5 mM to about 100 mM in the mixture comprising the sample to be treated, the denaturing solution, and the reducing solution.

32. The method according to claim 1, wherein said denaturing solution and reducing solution further comprises a mixture of sarcosyl, sodium dodecylsulfate and dithiothreitol.

33. The method according to claim 1, wherein the concentration of the chaotropic agent in the denaturing solution and reducing solution is about 1 M or greater.

34. The method according to claim 1, wherein the chaotropic agent is urea.

35. The method according to claim 1 further comprising, at step b) the denatured and reduced PrP is immobilized via a ligand which is a capture antibody capable of retaining the PrP by affinity binding, and at step c) the detection antibody is an antibody specifically binding to the PrP having a particular allelic form at position 171, this position being different from the epitopic site recognized by the capture antibody.

36. The method according to claim 1 further comprising, at step b) the denatured and reduced PrP is immobilized via a ligand which is a capture antibody specifically binding to the PrP having a particular allelic form at position 171, and at step c) the detection antibody is an antibody capable of binding to the PrP by affinity binding to an epitopic site different from the one recognized by the capture antibody.

37. The method according to claim 1 further comprising, at step b) the denatured and reduced PrP is immobilized via a ligand chosen from among plasminogens, avidine, streptavidine, glycose aminoglycans, hesperidine, porphyrins, streptamycine and tetracycline, and at step c) the detection antibody is an antibody specifically binding to the PrP having a particular allelic form at position 171.

38. The method according to claim 1 further comprising, at step b) the denatured and reduced PrP is immobilized directly on the solid phase, and at step c) the detection antibody is an antibody specifically binding to the PrP having a particular allelic form at position 171.

39. The method according to claim 1, wherein the solid phase is selected from the group consisting of microtiter plates, beads, tubes, in polymer, in polystyrene, in polyethylene, or in latex, and a microtiter plate in polystyrene.

40. The method according to claim 1, wherein the sample of biological fluid is blood, plasma, serum, or milk.

41. The method according to claim 1, wherein the ligand is a capture antibody is selected from the group consisting of antibodies SAF-15, SAF-31, SAF-32, SAF-33, SAF-34, SAF-35, SAF-37, 3B5, SAF-84, SHA-31, BAR-222, BAR-224, BAR-233, and 8G8.

42. The method according to claim 1, wherein the detection antibody is selected from the group consisting of the labeled 2A11 antibody, labeled 11C6 antibody, labeled BAR226 antibody, and the labeled antibody 12F10.

43. The method according to claim 35, wherein the capture antibody is the SAF-34 antibody or 3B5 antibody, and the detection antibody is the labeled 2A11 antibody.

44. The method according to claim 1, wherein the ligand is a capture antibody selected from the group consisting of antibodies 2A11, 11C6, BAR-226, and 12F10.

45. The method according to claim 1, wherein the detection antibody is selected from the group consisting of SAF-15, SAF-31, SAF-32, SAF-33, SAF-34, SAF-35, SAF-37, 3B5, SAF-84, SHA-31, BAR-222, BAR-224, BAR-233, and 8G8.

46. The method according to claim 36, wherein the capture antibody is selected from the group consisting of antibodies 2A11, 12F10, BAR226, and 11C6, and in that the detection antibody is selected from the group consisting of the labeled antibodies SAF-15, SAF-31, SAF-32, SAF-33, SAF-34, SAF-35, SAF-37, 3B5, SAF-84, SHA-31, BAR-222, BAR-224, BAR-233, and 8G8.

47. The method of claim 1, wherein the method screens an ovine population in relation to its resistance to transmissible subacute spongiform encephalopathies.

48. The method of claim 1, wherein the method screens an ovine population in relation to its sensitivity to transmissible subacute spongiform encephalopathies.

49. A kit to identify the genotype at position 171 of the ovine PrP, comprising:
a solid phase on which at least one capture antibody is immobilized, and selected from the group consisting of antibodies SAF-15, SAF-31, SAF-32, SAF-33, SAF-34, SAF-35, SAF-37, 3B5, SAF-84, SHA-31, BAR-222, BAR-224, BAR-233, and 8G8;
a denaturing and reducing solution; and
at least one detection antibody, selected from the group consisting of labeled antibodies 12F10, BAR226, 11C6, and 2A11, and more particularly the labeled antibody 2A11.

50. A kit to identify the genotype at position 171 of the ovine PrP, the kit comprising:
a solid phase on which at least one capture antibody is immobilized, and is selected from the group consisting of antibodies 12F10, BAR226, 11C6, and 2A11;
a denaturing and reducing solution; and
at least one detection antibody, is selected from the group consisting of labeled antibodies SAF-15, SAF-31, SAF-32, SAF-33, SAF-34, SAF-35, SAF-37, 3B5, SAF-84, SHA-31, BAR-222, BAR-224, BAR-233, and 8G8.
